(19) United States
(12) Patent Application Publication
(10) Pub. No.: US 2012/0065741 A1
(43) Pub. Date: Mar. 15, 2012

(54) GUIDED TISSUE REGENERATION MEMBRANE

(76) Inventors: Chao-Fu Chang, Chung-Li (CN); Kent Kuohua Chang, Cupertino, CA (US)

(21) Appl. No.: 12/880,502

(22) Filed: Sep. 13, 2010

(51) Int. Cl. A61F 2/02

(52) U.S. Cl. 623/23.72

(57) ABSTRACT

A guided tissue regeneration membrane has a top surface, a bottom surface and a plurality of through holes formed through the top and bottom surfaces. Each of the plurality of through holes has a base opening on the top surface and a tip opening on the bottom surface. The diameter of the base opening is larger than that of the tip opening. The guided tissue regeneration membrane is placed between a hard tissue and a soft tissue of gums with the top surface thereof facing the hard tissue so as to hinder the soft tissue from rapidly growing. The tip openings are available for the soft tissue to supply nutrient to the hard tissue therethrough. The hard tissue can grow from the base openings, through the corresponding through holes and to the soft tissue to repair periodontal tissue.

### BACKGROUND OF THE INVENTION

1. Field of the Invention

The present invention is related to a guided tissue regeneration membrane, and more particularly to a guided tissue regeneration membrane applied to periodontal tissue regeneration.

2. Description of the Related Art

Gum disease generally refers to a disease of periodontal ligament and alveolar bone. Normally, to prevent gums from swelling and reduce inflammation, the treatment of gum disease is to remove soft tissue (gums and part of periodontal ligament). However, after removing the soft tissue, the soft tissue grows faster than hard tissue, leading to root coverage by gums Loose teeth then appear because the growing speed of the hard tissue (alveolar bone) is not fast enough to support roots of teeth.

In the healing process of the periodontium, the repaired cells come from epithelium cells of gums, connective tissue in gums, bone cells inside alveolar bone and undifferentiated mesenchymal cells in periodontal ligament. Among them, epithelium cells grows much faster relative to others. Hence, the areas for gums are occupied by epithelium cells. In that case, periodontium can never recover to the original condition, and alveolar bone and periodontal ligament are unable to be fully repaired.

With reference to FIGS. 4 to 6, a conventional guided tissue regeneration technique first adequately cleans up a misfit 71 formed among gums 60, an alveolar bone 70 and a tooth 50, fills bone powder 90 in the misfit 71, places and properly fixes a guided tissue regeneration membrane 40 between the gums 60 and the alveolar bone 70 so that the guided regeneration membrane 40 can adequately isolate epithelium cells and connective tissue in gums Hence, bone cells in the alveolar bone 70 and undifferentiated mesenchymal cells in the periodontal ligament have enough time to grow and recover to health gum tissue.

Whereas, with reference to FIG. 7, both sides of the guided regeneration membrane 40 abut the soft tissue and the hard tissue. A thrust generated by the epithelium cells in the soft tissue acting upon the guided regeneration membrane 40 compresses a growing space of the hard tissue when the epithelium cells in the soft tissue grows in a relatively faster speed. Consequently, a good treatment result cannot be fulfilled.

### SUMMARY OF THE INVENTION

An objective of the present invention is to provide a guided tissue regeneration membrane applied to periodontal tissue regeneration.

To achieve the foregoing objective, the guided tissue regeneration membrane has a top surface, a bottom surface and a plurality of through holes.

The plurality of through holes are formed through the top surface and the bottom surface and each of the plurality of through holes defines a tip opening and a base opening. The tip opening is formed through the bottom surface. The base opening is formed through the top surface and has a diameter larger than that of the tip opening.

In accordance with the present invention, the guided tissue regeneration membrane is formed by a material that is characterized by cell occlusivity, tissue integration, biocompatibility, clinical manageability and spacemaking nature and includes but not limited to a group consisting of metal, polylactic acid, chitosan and collagen, or is formed by materials selected from a combination of the group.

The approach of the present invention is that the guided tissue regeneration membrane is applied between gums and an alveolar bone provided that the top surface of the membrane faces a periodontal hard tissue. Given a structure like this, the regeneration membrane blocks a soft tissue with its bottom surface while the soft tissue can still supply nutrients to the hard tissue through the plurality of tip openings. The space defined by each of the plurality of through holes taking a conic form facilitates the hard tissue having a slower growing speed relative to the soft tissue to grow and expand through the corresponding base opening having a larger diameter than that of the tip opening. Accordingly, the alveolar bone and the periodontal tissue of the hard tissue have enough space for proliferation in generation of new periodontal ligament and alveolar bone on roots of teeth and the purpose of completely regenerating periodontal tissue can be achieved.

### BRIEF DESCRIPTION OF THE DRAWINGS

FIG. 1 is a perspective view of a guided tissue regeneration membrane in accordance with the present invention;

FIG. 2 is a cross-sectional view of the guided tissue regeneration membrane in FIG. 1;

FIG. 3 is a cross-sectional view of the guided tissue regeneration membrane in FIG. 1 adopted to an application; and

FIGS. 4 to 7 are cross-sectional views illustrating a conventional guided tissue regeneration technique.

### DETAILED DESCRIPTION OF THE INVENTION

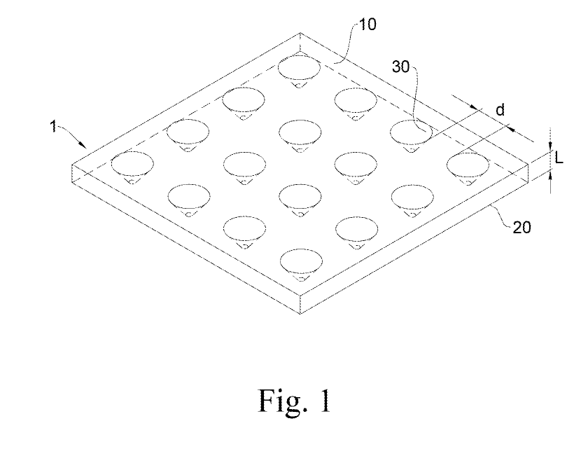

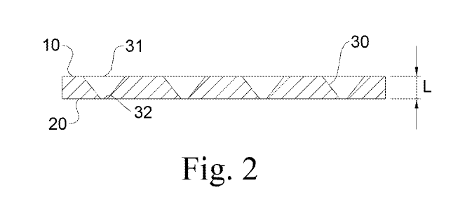

With reference to FIGS. 1 and 2, a guided tissue regeneration membrane in accordance with the present invention takes a form of a flake and has a top surface 10, a bottom surface 20 and a plurality of through holes 30. Each of the plurality of through holes 30 is conic, is formed through the top surface 10 and the bottom surface 20, and has a base opening 31 on the top surface 10 and a tip opening 32 on the bottom surface 20. The diameter of the base opening 31 is larger than that of the tip opening 32.

In the present embodiment, the guided tissue regeneration membrane 1 is formed by a material selected from a group consisting of metal, polylatic acid, chitosan, collagen, starch, ganoderma lucidum and a combination thereof. The guided tissue regeneration membrane 1 is molded by pressing a die having a plurality of protrusions on the guided tissue regeneration membrane 1. Each of the protrusions matches the corresponding through hole and may be a cone.

In the present embodiment, a thickness between the top surface 10 and the bottom surface 20 ranges from 0.1 mm to 3 mm, and preferably ranges from 0.5 mm to 0.1 mm. A distance between any two of the adjacent base openings 31 ranges from 50 nm to 50 μm. A diameter of each of the plurality of base opening 31 ranges from 100 μm and 1000 μm, and preferably ranges from 200 μm to 500 μm. A diameter of each of the plurality of tip openings 32 ranges from 1 nm to 100 μm, and preferably ranges from 50 nm to 50 μm.

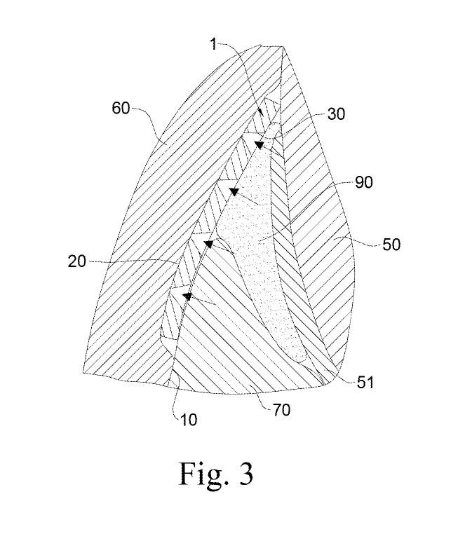

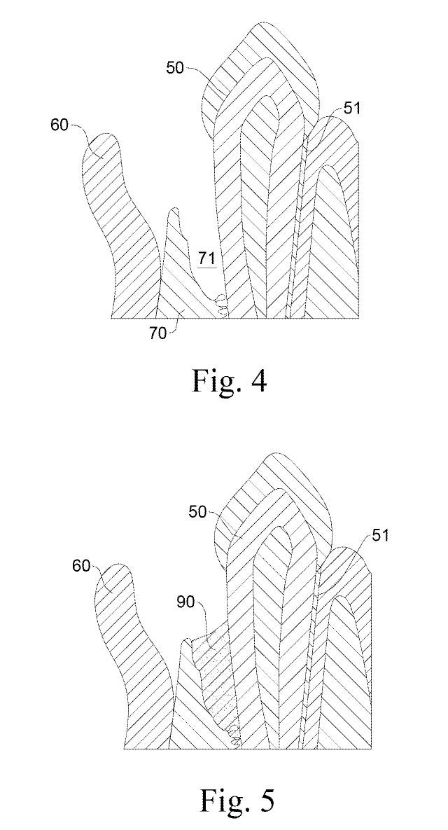

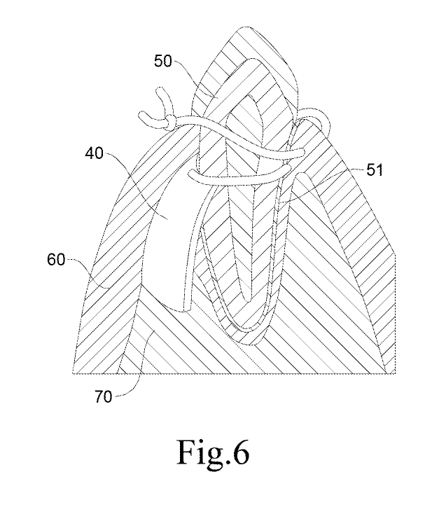

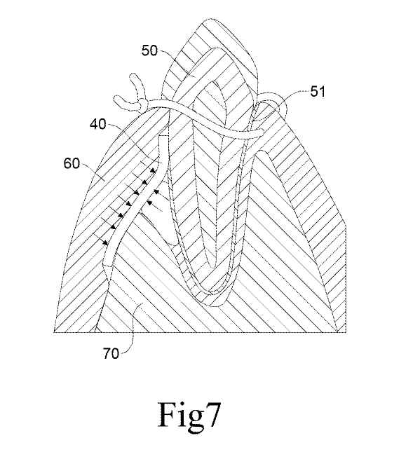

With reference to FIG. 3, when exercised, the guided tissue regeneration membrane 1 is placed among an alveolar bone 70, filled bone powder 90 and gums 60 so that the top surface 10 facing the alveolar bone 70 makes the base openings 31 on the top surface 10 abut the alveolar bone 70 and the filled bone powder 90. Meanwhile, the bottom surface 20 abuts the gums 60.

Because of the space delimited by the through holes 30 of the guided tissue regeneration membrane 1, the hard tissue having a slower growing speed relative to the epithelium cells inside the soft tissue can grow and expand into the space through the base openings so that the alveolar bone and periodontal tissue of the hard tissue can proliferate and the soft tissue can also supply the nutrients required for growth of the hard tissue through the tip openings 32 simultaneously. Accordingly, new periodontal ligament 51 and new alveolar bone can be formed on the surface of roots of teeth to achieve the purpose of tissue regeneration.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A guided tissue regeneration membrane, comprising:
a top surface;
a bottom surface; and
a plurality of through holes formed through the top surface and the bottom surface and each of the plurality of through holes defining:
a tip opening formed through the bottom surface; and
a base opening formed through the top surface and having a diameter larger than that of the tip opening.

2. The guided tissue regeneration membrane as claimed in claim 1, formed by a material selected from a group consisting of metal, polylatic acid, chitosan, collagen, starch, ganoderma lucidum and a combination thereof.

3. The guided tissue regeneration membrane as claimed in claim 1, wherein each of the plurality of through holes is conic.

4. The guided tissue regeneration membrane as claimed in claim 1, wherein a thickness between the top surface and the bottom surface ranges from 0.1 mm to 3 mm.

5. The guided tissue regeneration membrane as claimed in claim 1, wherein a distance between any two of the adjacent base openings ranges from 50 nm to 50 μm.

6. The guided tissue regeneration membrane as claimed in claim 1, wherein a diameter of each of the plurality of base openings ranges from 100 μm to 1000 μm.

7. The guided tissue regeneration membrane as claimed in claim 2, wherein a diameter of each of the plurality of base openings ranges from 100 μm to 1000 μm.

8. The guided tissue regeneration membrane as claimed in claim 3, wherein a diameter of each of the plurality of base openings ranges from 100 μm to 1000 μm.

9. The guided tissue regeneration membrane as claimed in claim 4, wherein a diameter of each of the plurality of base openings ranges from 100 μm to 1000 μm.

10. The guided tissue regeneration membrane as claimed in claim 5, wherein a diameter of each of the plurality of base openings ranges from 100 μm to 1000 μm.

11. The guided tissue regeneration membrane as claimed in claim 1, wherein a diameter of each of the plurality of tip openings ranges from 1 nm to 100 μm.

12. The guided tissue regeneration membrane as claimed in claim 2, wherein a diameter of each of the plurality of tip openings ranges from 1 nm to 100 μm.

13. The guided tissue regeneration membrane as claimed in claim 3, wherein a diameter of each of the plurality of tip openings ranges from 1 nm to 100 μm.

14. The guided tissue regeneration membrane as claimed in claim 4, wherein a diameter of each of the plurality of tip openings ranges from 1 nm to 100 μm.

15. The guided tissue regeneration membrane as claimed in claim 5, wherein a diameter of each of the plurality of tip openings ranges from 1 nm to 100 μm.

16. The guided tissue regeneration membrane as claimed in claim 6, wherein a diameter of each of the plurality of tip openings ranges from 1 nm to 100 μm.

17. The guided tissue regeneration membrane as claimed in claim 7, wherein a diameter of each of the plurality of tip openings ranges from 1 nm to 100 μm.

18. The guided tissue regeneration membrane as claimed in claim 8, wherein a diameter of each of the plurality of tip openings ranges from 1 nm to 100 μm.

19. The guided tissue regeneration membrane as claimed in claim 9, wherein a diameter of each of the plurality of tip openings ranges from 1 nm to 100 μm.

20. The guided tissue regeneration membrane as claimed in claim 10, wherein a diameter of each of the plurality of tip openings ranges from 1 nm to 100 μm.
